(19) United States
(12) Patent Application Publication
(10) Pub. No.: US 2003/0203964 A1
(43) Pub. Date: Oct. 30, 2003

(54) Process for producing medicaments which comprise HMG CoA reductase inhibitors

(76) Inventors: Fritz Schuckler, Bergisch Gladbach (DE); Karl Geisen, Koln (DE); Norbert Pollinger, Mullheim (DE); Samir Samaan, Wuppertal (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/456,222

(22) Filed: Jun. 5, 2003

Related U.S. Application Data
(63) Continuation of application No. 09/446,342, filed on Dec. 16, 1999, now abandoned.
Application No. 09/446,342 is a 371 of international application No. PCT/EP98/03294, filed on Jun. 3, 1998.

(30) Foreign Application Priority Data
Jun. 16, 1997 (DE) 197 25 391.1

(51) Int. Cl. A61K 31/366; A61K 31/22; A61K 31/19

(52) U.S. Cl. 514/460; 514/546; 514/557

(57) ABSTRACT

The invention relates to a process for producing medicaments which comprise HMG CoA reductase inhibitors in the form of their salts, characterized in that the corresponding precursors of the actual active compounds are converted with bases, which contain alkali metal or alkaline earth metal ions, in a solvent, into the active compound, and the resulting active compound-containing solution is processed into the desired administration form.

The invention relates to a process for producing medicaments which comprise HMG CoA reductase inhibitors and which are, in particular, in the form of granules, tablets and pellets.

It is known that HMG CoA reductase inhibitors are employed as active compounds in medicaments for treating hyperlipoproteinaemia and arteriosclerosis. Most of these active compounds are derived from the statin group, having the following formula

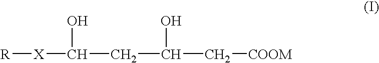

in which

R represents an organic radical,

X represents a group —CH₂—CH₂— or —CH═CH—; in particular in the (E) form, and

M represents a physiologically acceptable cation, for example from the alkali metal cation group, preferably sodium or potassium, and also represents an ammonium ion.

It is also known that most active compounds of the statin group are used in the form of their salts. For this, these active compounds are usually first of all prepared, in aqueous solution, from the corresponding esters or acids, as precursors, by treating with bases, and this solution is then lyophilized in order to obtain the actual active compound (EP 547 000).

By its nature, this process is very time-consuming and cost-intensive and requires, inter alia, a substantial input in terms of process monitoring, control and optimization. In addition to this, a lyophilization product is very difficult to handle since it exhibits very strong hygroscopic properties. This can result in substantial problems in storage and production (e.g. inaccuracy in weighing, and variations in drug content in the tablets due to moisture uptake in the granular state) despite careful adherence to climatically favourable conditions (low atmospheric humidity) or despite using special moisture-tight packing materials.

A process has now been found for producing medicaments which comprise HMG CoA reductase inhibitors of the statin group having the general formula (I)

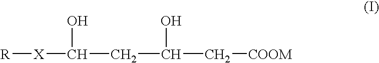

in which

R represents an organic radical,

X represents a group —CH₂—CH₂— or —CH═CH—, and

M represents a pharmacologically acceptable cation,

characterized in that the actual active compound is first of all prepared, in aqueous solution/suspension, from the corresponding active compound precursor by treating the latter with an aqueous base, and this active compound-containing solution/suspension is then either directly sprayed onto excipients, and dried in parallel with this, or granulated after mixing the active compound-containing solution/suspension with suitable binders and excipients, and then dried.

The novel process consequently describes the production of medicaments without the active compound being isolated in its solid form or as a pure substance but, instead, being directly subjected to further processing as a solution.

This considerably simplifies the entire production process, particularly because the problematical and expensive lyophilization step for isolating the active compound is dispensed with. This also brings about a substantial improvement in the manageability and productive reliability of the medicinal preparation.

In general, those bases which contain alkali metal and alkaline earth metal ions are used as bases. Preference is given to hydroxides, carbonates or hydrogen carbonates which contain alkali metal or alkaline earth metal ions, such as sodium, potassium, lithium, beryllium, calcium or magnesium ions. Particular preference is given to using sodium hydroxide or potassium hydroxide, depending on whether the actual active compound is a sodium salt or a potassium salt.

The quantity of base is at least an equimolar quantity in relation to the quantity of active compound precursor employed.

In this context, precursors are understood as meaning the corresponding acids or esters of the formula (II)

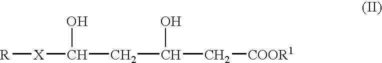

or the corresponding lactones of the formula (III)

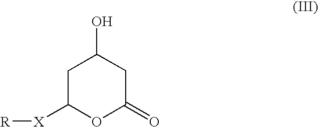

in which

R represents an organic radical,

X represents a group of the formula —CH₂—CH₂— or —CH═CH—, and

R¹ represents a C₁-C₄-alkyl group or hydrogen.

Preference is given to employing the lactones of the formula (III) as precursors, with the actual active compounds being prepared by cleaving the lactone ring with the base directly in the solution.

Cerivastatin lactone, which is converted, in solution, with sodium hydroxide into the active compound cerivastatin, is particularly preferably suitable.

The novel process is suitable for producing solid and liquid, in particular solid medicinal forms, such as active compound-containing powders, granules, tablets or pellets. In this connection, it is possible to compress the powders, granules or pellets into tablets or to fill them into capsules.

As binding agents for the granulation, all the customary pharmaceutically acceptable binding agents can be used, e.g. polyvinylpyrrolidones, gelatin and starch and cellulose derivatives (natural or synthetic). Polyvinylpyrrolidones, e.g. polyvinylpyrrolidone 25, are preferred.

All the customary pharmaceutical excipients can be used as additional auxiliary substances, thus cellulose derivatives (e.g. microcrystalline cellulose), sugars (e.g. lactose), sugar alcohols (e.g. mannitol or sorbitol) and inorganic fillers (e.g. calcium phosphates) can, for example, be used as fillers, and use can also be made of all the other excipients which are required for producing medicinal formulations having the desired properties, e.g. lubricants (e.g. magnesium stearate), e.g. disintegrants (e.g. crosslinked polyvinylpyrrolidone or sodium carboxymethyl cellulose), e.g. wetting agents (e.g. sodium lauryl sulphate), e.g. stabilizers, e.g. fragrances and e.g. dye pigments.

Lactose, mannitol and microcrystalline cellulose are preferably used as fillers.

he proportion of binding agent in the total mixture is preferably from 0 to 20%. The proportion of fillers and auxiliary substances in the total mixture is preferably from 70 to 99%.

The drying temperature is generally from 40 to 120° C., preferably from 60 to 100° C.

The novel process is particularly suitable for active compounds of the formula (I) in which the substituent R denotes an optionally substituted pyrimidine, indole, indolizine, pyrrolopyridine, quinoline, dihydroquinoline, pyrazolopyridine, pyridazine, imidazole, pyrroloisoquinoline, pyridine, pyrrole or tetrazole radical.

The novel process is particularly suitable for the following active compounds of the formula (I):

pravastatin,

3R,5S-(E)-7-[4-(4-fluorophenyl)-6-(1-methylethyl)-2-dimethylaminopyrimidine-5-yl]-3,5-dihydroxy-6-heptenoic acid, sodium salt;

erythro-(±)-(E)-7-[3-(4-fluorophenyl)-spiro[cyclopentane-1,1¢-1H-inden]-2¢-yl]-3,5-dihydroxy-6-heptenoic acid, sodium salt;

3R,5S-(E)-7-[3-(4-fluorophenyl)-1-(1-methylethyl)-indolizin-3-yl]-3,5-dihydroxy-6-heptenoic acid, sodium salt;

3R,5S-(E)-7-[3-(4-fluorophenyl)-1-(1-methylethyl)-1H-pyrrolo[2,3-b]pyridin-2-yl]-3,5-dihydroxy-8-heptenoic acid, sodium salt;

3R,5S-(E)-7-[4-(4-fluorophenyl)-2-(1-methylethyl)-quinolin-3-yl]-3,5-dihydroxy-6-heptenoic acid, sodium salt;

3R,5S-(E)-7-[1-(4-fluorophenyl)-3-(1-methylethyl)-4-oxo-1,4-dihydroquinolin-2-yl]-3,5-dihydroxy-6-heptenoic acid, sodium salt;

3R,5S-(E)-7-[4-(4-fluorophenyl)-6-(1-methylethyl)-3-methyl-1H-pyrazolo[3,4-b]pyridin-5-yl]-3,5-dihydroxy-6-heptenoic acid, sodium salt;

3R,5S-(E)-7-(3-(1-methylethyl)-5,6-diphenyl-pyridazin-4-yl]-3,5-dihydroxy-6-heptenoic acid, sodium salt;

3R-5S-(E)-7-[4-(4-fluorophenyl)-6-(1-methylethyl)-2-phenyl-pyrimidin-5-yl]-3,5-dihydroxy-6-heptenoic acid, sodium salt;

3R,5S-(E)-7-[4-(4-fluorophenyl)-1-(1-methylethyl)-3-phenyl-2-oxo-2,3-dihydroimidazol-5-yl]-3,5-dihydroxy-6-heptenoic acid, sodium salt;

3R, 5S-(E)-7-[4-(4-fluorophenyl)-2-(1-methylethyl)-1-oxo-1,2-dihydro-quinolin-3-yl]-3,5-dihydroxy-6-heptenoic acid, sodium salt;

erythro-(±)-(E)-7-[4-(4-fluorophenyl)-2-(1-methylethyl)-quinolin-3-yl]-3,5-dihydroxy-6-heptenoic acid, sodium salt;

erythro-(±)-(E)-7-[1-(4-fluorophenyl)-3-(1-methylethyl)-pyrrolo-[2,1-a]isoquinolin-2-yl]1-3,5-dihydroxy-6-heptenoic acid, sodium salt;

erythro-(±)-(E)-7-[4-cyclopropyl-6-(4-fluorophenyl)-2-(4-methoxyphenyl)pyrimidin-5-yl]-3,5-dihydroxy-6-heptenoic acid, sodium salt;

3R,5S-(E)-7-[4-(4-fluorophenyl)-2,6-dimethylpyrimidin-5-yl]-3,5-dihydroxy-8-heptenoic acid, sodium salt;

3R,5S-(E)-7-[4-(4-fluorophenyl)-6-methyl-2-phenyl-pyrimidin-5-yl]-3,5-dihydroxy-8-heptenoic acid, sodium salt;

3R,5S-(E)-7-[4-(3,5-dimethylphenyl)-6-methyl-2-phenyl-pyrimidin-5-yl]-3,5-dihydroxy-6-heptenoic acid, sodium salt;

erythro-(±)-(E)-7-[3,4-bis(4-fluorophenyl)-6-(1-methylethyl)-pyridazin-5-yl]-3,5-dihydroxy-6-heptenoic acid, sodium salt;

erythro-(±)-(E)-7-[1-(4-fluorophenyl)-3-(1-methylethyl)-5-phenyl-1H-pyrrol-2-yl]-3,5-dihydroxy-6-heptenoic acid, sodium salt;

erythro-(±)-(E)-9,9-bis(4-fluorophenyl)-3,5-dihydroxy-8-(1-methyl-1H-tetrazol-5-yl)-6,8-nonadienoic acid, sodium salt;

erythro-(±)-(E)-3,5-dihydroxy-9,9-diphenyl-6,8-heptenoic acid, sodium salt;

erythro-(±)-(E)-7-[4-(4-fluorophenyl)-1,2-bis(1-methylethyl)-3-phenylpyrrol-2-yl]-3,5-dihydroxy-6-heptenoic acid, sodium salt;

3R,5S-(E)-7-[4,5-bis(4-fluorophenyl)-2-(1-methylethyl)-1H-imidazol-1-yl]-3,5-dihydroxy-6-heptenoic acid, sodium salt;

3R,5S-(E)-7-[4-(4-fluorophenyl)-2,6-bis(1-methylethyl)-5-methoxymethyl-pyuidin-3-yl]-3,5-dihydroxy-6-heptenoic acid, sodium salt; (cerivastatin)

erythro-(±)-(E)-[4-(4-fluorophenyl)-2-(1-methylethyl)-6-phenyl-pyridin-3-yl]-3,5-dihydroxy-6-heptenoic acid, sodium salt;

erythro-(±)-(E)-[2-(4-fluorophenyl)-4,4,6,6-tetramethyl-cyclohexen-1-yl]-3,5-dihydroxy-6-heptenoic acid, sodium salt;

erythro-(±)-(E)-7-[4-(4-fluorophenyl)-2-cyclopropyl-quinolin-3-yl]-3,5-dihydroxy-6-heptenoic acid, sodium salt; and

erythro-(±)-(E)-7-[4-(4-fluorophenyl)-2-(1-methylethyl)-quinolin-3-yl]-3,5-dihydroxy-6-heptenoic acid, sodium salt.

The novel process is particularly suitable for 3R,5S-(E)-7-[4-(4-fluorophenyl)-2,6-bis(1-methylethyl)-5-methoxymethyl-pyridin-3-yl]-3,5-dihydroxy-6-heptenoic acid, sodium salt.

In addition, the described process is particularly suitable when the active compound is only being employed in very small quantities, e.g. less than 5%, preferably less than 1% (proportion by weight in the final formulation). By processing the solution or suspension of active compound into the granulation liquid and then coating the carrier, medicinal preparations can be produced in which the distribution of the active compound is exceptionally uniform. The well known problems which arise in association with the conventional (dry) mixing of components which are present in a total mixture in very different proportions are thereby avoided in a simple manner.

### EXAMPLES

### Example 1

A defined quantity of the active compound precursor cerivastatin pyridine lactone is initially introduced into a suitable vessel. The quantity of sodium hydroxide which is required for the quantitative conversion to cerivastatin, which quantity is calculated as a molar ratio between cerivastatin pyridine lactone and sodium hydroxide and which is in the form of an aqueous solution, e.g. 2.5% (w/w), and also a further calculated excess of water, e.g. 6.6 times the weight of cerivastatin pyridine lactone taken, are combined and added to the solid. The mixture is maintained under suitable conditions for a defined time, during which the precursor is converted into the active compound cerivastatin. The completeness of the conversion is checked.

After the transformation has come to an end, the mixture is filtered and polyvinyl-pyrrolidone 25 and water are then added. The following composition may be mentioned by way of example (values in [mg] in relation to the final formulation):

| | |
|---|---|
| Cerivastatin | 0.1 |
| Polyvinylpyrrolidone 25 | 1.8 |
| Water | q.s. |

The resulting granulation liquid is applied directly to a suitable carrier material, e.g. mannitol, a procedure which can be performed, in accordance with the current state of the art, either as a mixer granulation, e.g. with the aid of a high shear mixer, or as a fluidized bed granulation (value in [mg] in relation to the final formulation):

| | |
|---|---|
| Mannitol | 83.95 |

After drying, the batch is sieved and mixed.

The resulting granules can, for example, also be mixed, after the sieving, in the added presence of suitable lubricants (e.g. magnesium stearate) and disintegrants (e.g. crosslinked polyvinylpyrrolidone), then processed into tablets (weight, 90 mg, diameter, 6 mm) and subsequently lacquered (protecting the active compound against light).

### Example 2

Like Example 1, except that the liquid is not granulated on a pulverulent carrier and is instead absorbed onto pellets in suitable apparatus, for example fluidized bed equipment fitted with a Wurster insert.

### Example 3

Like Example 1, except that the powder vehicle is rounded off into active compound-containing pellets in suitable apparatus, for example fluidized bed units, rotary granulators or similar equipment.

### Example 4

Like Example 1,except that the liquid is absorbed onto the pulverulent support material in powder coaters, with active compound-coated powders being produced.

### Example 5

Like Examples 1 to 4, except that the active compound-coated powder, the granules or the pellets are filled into premade capsules, which are for example composed of hard gelatine or other suitable materials.

### Example 6

Like Example 1, except that the concentration of the active compound per dose unit is decreased to 0.01 mg or increased to 5.0 mg.

### Example 7

Like Example 1, except that a stabilizer, e.g. sodium hydroxide, is added to the granulation liquid.

### Example 8

Like Example 1, except that the stabilizer, e.g. sodium carbonate, is added to the support material.

### Example 9

For a liquid formulation (aqueous) with the option of, if necessary, back-titrating (adjusting, since alkaline after hydrolysis) the pH.

1. Process for producing medicaments which comprise HMG CoA reductase inhibitors of the stating group having the general formula I 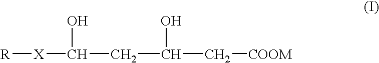
in which

| | |
|---|---|
| R | represents an organic radical, |
| X | represents a group —CH₂—CH₂— or —CH═CH—, and |
| M | represents a pharmacologically acceptable cation, |

characterized in that the actual active compound is prepared in aqueous solution/suspension by treating a corresponding active compound precursor with an aqueous base, and this active compound-containing solution/suspension is then either directly sprayed onto neutral excipients, without isolating the active compound as a solid substance, and dried in parallel with this, or, after mixing the active compound-containing solution with suitable binders, is then granulated with excipients and subsequently dried.

2. Process according to claim 1, characterized in that the acids or esters 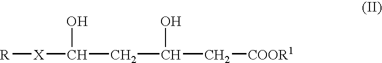
or the corresponding lactones of the formula (III) 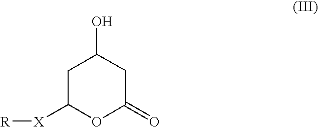
in which
R represents an organic radical,
X represents a group of the formula —CH₂—CH₂— or —CH═CH—, and
R¹ represents a C₁-C₄-alkyl group or hydrogen, are employed as precursors.

3. Process according to claims 1 and 2, characterized in that sodium hydroxide or potassium hydroxide is employed as base.

4. Process according to claims 1 to 3, characterized in that polyvinylpyrrolidone, gelatin or starch and cellulose derivatives is/are employed as binders.

5. Process according to claims 1 to 4, characterized in that cellulose derivatives, sugars, sugar alcohols or inorganic fillers are employed as excipients.

6. Process according to claims 1 to 5, characterized in that mannitol or sorbitol is employed as excipients.

7. Process according to claims 1 to 6, characterized in that the proportion of binder in the total mixture is 0-20%.

8. Process according to claims 1 to 7, characterized in that the proportion of fillers and excipients in the total mixture is 70-99%.

9. Process according to claims 1 to 8, characterized in that at least an equimolar quantity of base, calculated in relation to the active compound precursor, is employed.

10. Process according to claims 1 to 9, characterized in that the quantity of active compound is less than 5% of the total quantity of the final medicinal form.
